(19) United States
(12) Patent Application Publication
(10) Pub. No.: US 2007/0167917 A1
(43) Pub. Date: Jul. 19, 2007

(54) Disposable syringe with a swab pack

(76) Inventor: David Hakmo Lee, Little Neck, NY (US)

Correspondence Address: David H. Lee, 34-15 255th ST, Little Neck, NY, 11363, US

(21) Appl. No.: 11/334,899

(22) Filed: Jan. 19, 2006

(51) Int. Cl. A61M 5/00

(52) U.S. Cl. 604/187

(57) ABSTRACT

A disposable syringe with an antiseptic liquid-dipped swab pack permits a syringe user to disinfect his or her body spot for injection with a self-contained antiseptic swab. The syringe comprises a hollow cylinder for containing liquid medication, a needle extending forwards from a frontal end of the cylinder, a plunger slidably fitted to the cylinder at an opposite side from the needle in a liquid-tight manner, a hermetically sealed flexible swab pack containing antiseptic liquid-dipped swab and removably held by the cylinder, and a fastener for keeping the swab pack in place on the cylinder against inadvertent removal.

### FIELD OF THE INVENTION

The present invention is directed to a disposable syringe with a swab pack and more specifically to a disposable syringe that carries an antiseptic liquid-dipped swab pack with it to allow a syringe user to disinfect his or her body spot for injection through the use of a self-contained antiseptic swab.

### BACKGROUND OF THE INVENTION

An increasing number of disposable syringes are used in hospitals and at homes in place of permanent use medication injectors, mainly because the former is less costly to purchase and requires no pre-injection sterilizing. Typically, the disposable syringes are composed of a hollow cylinder for containing medication liquid, a needle extending forwards from the frontal end of the cylinder and a plunger slidably fitted to the cylinder from the rear end thereof in a liquid-tight manner.

In order to inject medication into a body, the user should first prepare a medication container usually in the form of a bottle or ampoule, a disposable syringe, a liquid disinfectant agent such as alcohol, and a swab, whether dry or dipped with the liquid disinfectant agent. Then, a target body spot is rubbed with the wet swab for the purpose of disinfection, and the medication is injected into the body through the target spot by using the syringe, after which the needle-penetrated body spot is pressed with the swab for a while to stop bleeding and conduct disinfection once again.

Disinfecting the body spot before and after medication injection is an essential step that must be conducted to prevent bacteria infection and hence assure safety of the user. This means that, in addition to the syringe, disinfection products including an alcohol swab have to be further prepared in advance of medication injection. Absent the disinfection products, the syringe user should either give up injecting medication or push ahead with the medication injection in defiance of bacteria infection, which may jeopardize the user in terms of private sanitation.

Particularly, most of diabetics should administer an injection of insulin, a blood sugar reducing agent used in the treatment and control of diabetes mellitus, on a regular time basis. Unlike the typical injection made by nurses in hospitals, the insulin injection is hypodermically administered once or twice in a day, which hypodermic injection is far easier to make than an intravenous injection. In view of this, it has been the general trend that the diabetics or their family members who had some education regarding how to make insulin injection administer the injection of insulin by themselves at homes or other out-of-hospital places.

For the purpose of such insulin administration, the diabetics should separately prepare and store alcohol bottles and swabs for disinfection treatment, as well as the disposable syringes. In case of making a trip or going out for an extended period of time, the diabetics should carry the disinfection products and the syringes item by item. It goes without saying that acquiring, storing and carrying these injection articles on an item-by-item basis makes the user feel cumbersome and inconvenient. Furthermore, if the disinfection products are used up or missing inadvertently, the syringe user has no choice but to make injection of medication without disinfecting the needle-penetrated body spot, which may expose the user to a danger of bacteria infection.

Accordingly, a need has existed for a disposable syringe that has a self-contained antiseptic swab, thus eliminating the disadvantages noted above.

### SUMMARY OF THE INVENTION

It is an object of the present invention to provide disposable syringe with a swab pack that permits a syringe user to disinfect his or her body spot for injection through the use of a self-contained antiseptic swab, thereby eliminating the need to separately acquire, store and carry antiseptic products apart from a disposable syringe.

In accordance with one aspect of the invention, there is provided a disposable syringe, comprising: a hollow cylinder for containing liquid medication; a needle extending forwards from a frontal end of the cylinder; a plunger slidably fitted to the cylinder at an opposite side from the needle in a liquid-tight manner; a hermetically sealed flexible swab pack containing antiseptic liquid-dipped swab, the swab pack removably held by the cylinder; and a fastener means for keeping the swab pack in place on the cylinder against inadvertent removal.

According to another aspect of the invention, there is provided a disposable syringe, comprising: a hollow cylinder for containing liquid medication; a needle extending forwards from a frontal end of the cylinder; a plunger slidably fitted to the cylinder at an opposite side from the needle in a liquid-tight manner and having a grip exposed to the outside from the cylinder; a hermetically sealed flexible swab pack containing antiseptic liquid-dipped swab, the swab pack removably held by the grip of the plunger; and a fastener means for keeping the swab pack in place on the grip of the plunger against inadvertent removal.

### BRIEF DESCRIPTION OF THE DRAWINGS

The above and other objects, features and advantages of the present invention will become apparent from the following description of a preferred embodiment given in conjunction with the accompanying drawings, in which:

FIG. 1 is a perspective view showing a disposable syringe with a swab pack according to a first embodiment of the present invention;

FIG. 2 is an exploded perspective view of the disposable syringe shown in FIG. 1, illustrating a swab pack in an unrolled condition;

FIG. 3 is a perspective view showing one modification of the disposable syringe according to the first embodiment of the present invention;

FIG. 4 an exploded perspective view of the disposable syringe shown in FIG. 3, illustrating a swab pack in an unrolled condition;

FIG. 5 is a cross-sectional view taken along line—in FIG. 4;

FIG. 6 is a cross-sectional view showing a modified swab pack that can be employed in the disposable syringe illustrated in FIG. 3;

FIG. 7 is a perspective view showing another modification of the disposable syringe according to the first embodiment of the present invention;

FIG. 8 is a cross-sectional view taken along line—in FIG. 7;

FIG. 9 a perspective view showing a disposable syringe with a swab pack according to a second embodiment of the present invention;

FIG. 10 is a perspective view showing one modification of the disposable syringe according to the second embodiment of the present invention; and

FIG. 11 is a perspective view showing another modification of the disposable syringe according to the second embodiment of the present invention, wherein an end cap is used as a fastener means for a swab pack.

### DETAILED DESCRIPTION OF THE INVENTION

Preferred embodiments of the present invention will now be described in detail with reference to the accompanying drawings.

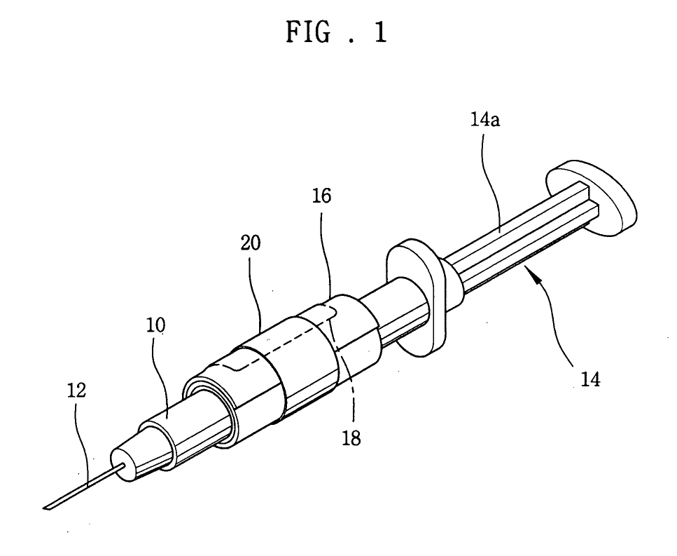

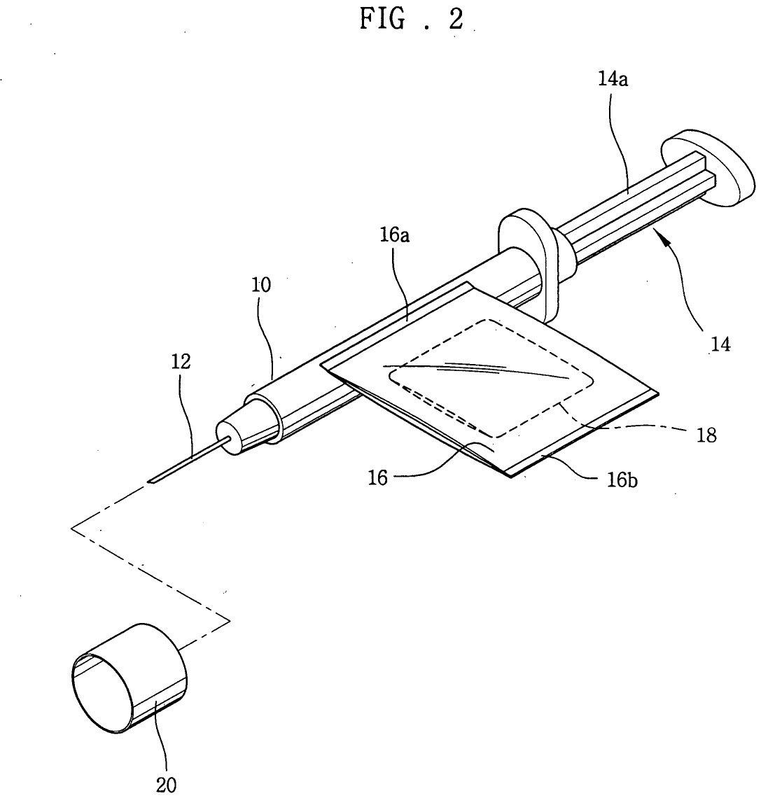

Referring first to FIGS. 1 through 2, there is shown a disposable syringe with a swab pack according to a first embodiment of the present invention. As shown, the disposable syringe is composed of a hollow cylinder 10 for containing liquid medication, e.g., insulin solution. A needle 12 extends forwards from the frontal end of the cylinder 10 and provides a flow path through which the liquid medication is injected into the body of a syringe user. A plunger 14 is slidably fitted to the cylinder 10 at an opposite side from the needle 12 in a liquid-tight manner. The plunger 14 has a grip 14a that can be grasped by the syringe user. The plunger 14 is movable between a retracted position in which the liquid medication is injected through the needle 12 and an extended position in which the liquid medication is drawn into the cylinder 10.

Removably held by the cylinder 10 is a hermetically sealed flexible swab pack 16 that contains a swab 18 dipped with antiseptic liquid such as alcohol. The swab pack 16 is made of, e.g., a synthetic resin film with air and liquid impermeability and hermetically sealed to avoid volatilization and leakage of the antiseptic liquid impregnated into the swab 18. The swab pack 16 is readily torn by the syringe user so that the swab can be taken out when needed, namely, when the body spot is to be rubbed with the swab for pre-injection and post-injection disinfection.

As best illustrated in FIG. 2, the swab pack 16 is of generally flat rectangular configuration and exhibits increased flexibility. The swab pack 16 is preferably wound around the cylinder 10 in a spiral shape, as shown in FIG. 1, although it may be folded to a small size and pressed against the cylinder 10. The swab pack 16 has a first end part 16a in contact with the cylinder 10 and a second end part 16b lying over an intermediate extension of the swab pack 16, when wound around the cylinder 10.

A fastener means is employed to keep the swab pack 16 in place on the cylinder 10 against inadvertent removal. A sheet sleeve 20 serves as the fastener means in the embodiment shown in FIGS. 1 and 2 and is adapted to encircle the swab pack 16 wound around the cylinder 10 to thereby keep the swab pack 16 in place. The sheet sleeve 20 is made of easy-to-tear materials, e.g., paper, such that the syringe user can tear away the sheet sleeve 20 in order to separate the swab pack 16 from the cylinder 10. In place of the sheet sleeve 20 of annular shape shown in FIGS. 1 and 2, it would be possible to wrap a planar paper sheet around the cylinder 10 and the swab pack 16 and have the opposite ends of the paper sheet bonded together.

When in use, the syringe user removes the sheet sleeve 20 by tearing or slipping so that the swab pack 16 can be unrolled from the cylinder 10. Then, the swab pack 16 is torn to expose the swab 18 to the outside, after which the user picks up the swab 18 and rubs the body spot with the swab 18 for disinfection treatment before and after injection of liquid medication. As used herein, the term “body spot” refers to a body area through which the needle 12 of the syringe is penetrated for medication injection.

The disposable syringe set forth above allows the syringe user to acquire, store and carry the syringe and the swab pack as a kit, thus eliminating the need to separately prepare them for medication injection.

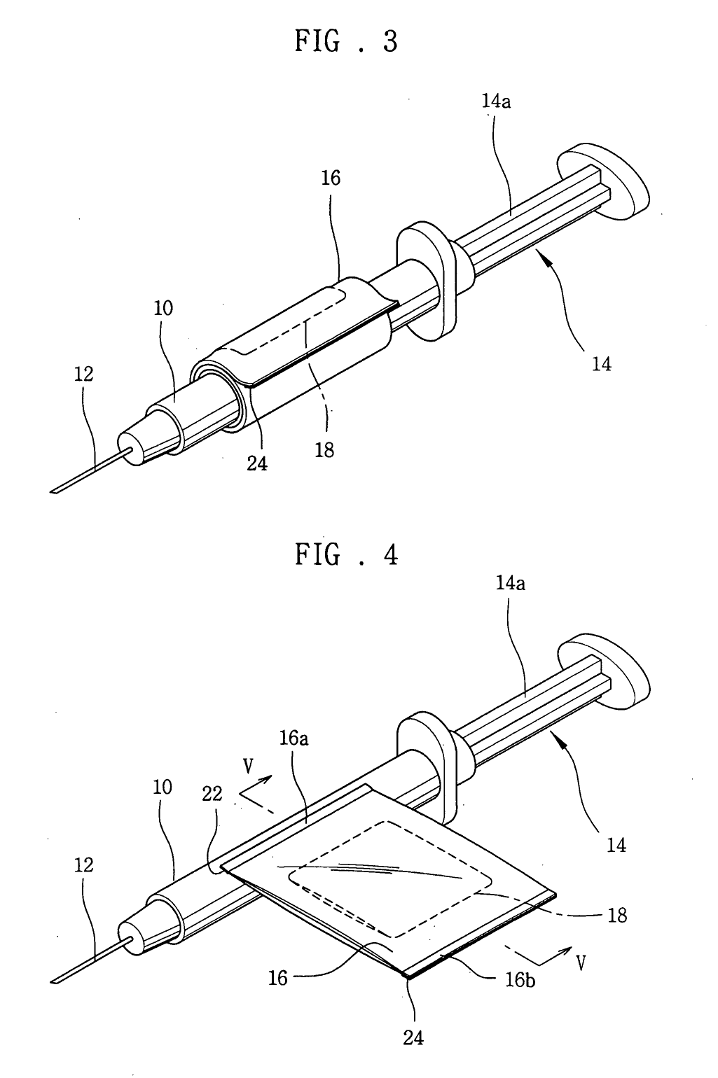

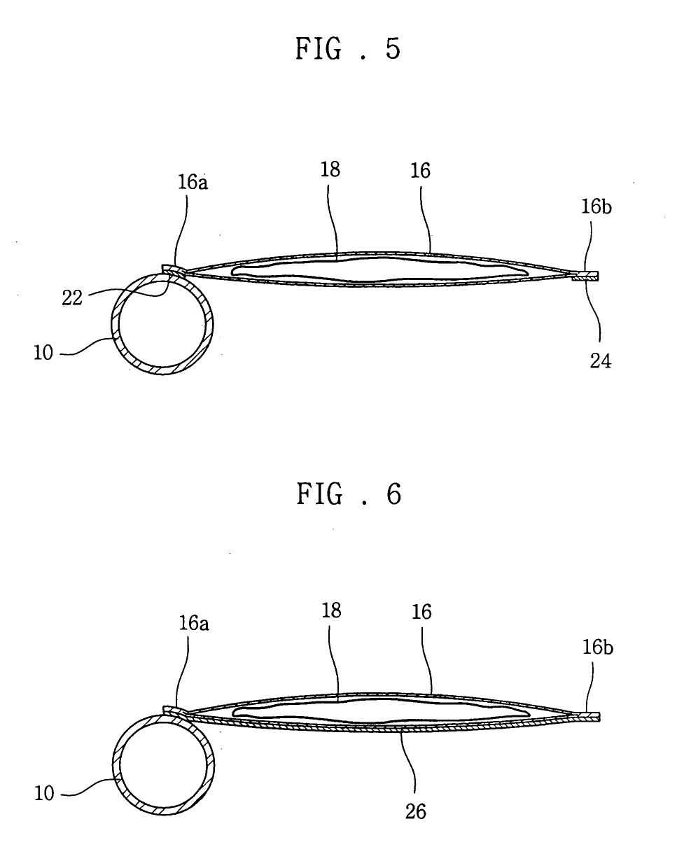

Turning to FIGS. 3 through 5, there is shown one modification of the disposable syringe according to the first embodiment of the present invention. As in the first embodiment describe above, the disposable syringe is composed of a hollow cylinder 10 for containing liquid medication, e.g., insulin solution. A needle 12 extends forwards from the frontal end of the cylinder 10 and provides a flow path through which the liquid medication is injected into the body of a syringe user. A plunger 14 is slidably fitted to the cylinder 10 at an opposite side from the needle 12 in a liquid-tight manner. The plunger 14 has a grip 14a that can be grasped by the syringe user. The plunger 14 is movable between a retracted position in which the liquid medication is injected through the needle 12 and an extended position in which the liquid medication is drawn into the cylinder 10.

Removably held by the cylinder 10 is a hermetically sealed flexible swab pack 16 that contains a swab 18 dipped with antiseptic liquid such as alcohol. The swab pack 16 is made of, e.g., a synthetic resin film with air and liquid impermeability and hermetically sealed to avoid volatilization and leakage of the antiseptic liquid impregnated into the swab 18. The swab pack 16 is readily torn by the syringe user so that the swab can be taken out when needed, namely, when the body spot is to be rubbed with the swab for pre-injection and post-injection disinfection.

As clearly shown in FIG. 4, the swab pack 16 is of generally flat rectangular configuration and exhibits increased flexibility. The swab pack 16 is preferably wound around the cylinder 10 in a spiral shape, as shown in FIG. 3, although it may be folded to a small size and pressed against the cylinder 10. The swab pack 16 has a first end part 16a in contact with the cylinder 10 and a second end part 16b lying over an intermediate extension of the swab pack 16, when wound around the cylinder 10.

A fastener means is employed to keep the swab pack 16 in place on the cylinder 10 against inadvertent removal. A first adhesive layer 22 and a second adhesive layer 24 serve as the fastener means in the modification shown in FIGS. 3 through 5. The first adhesive layer 22 is formed on one surface of the first end part 16a of the swab pack 16, viz., on the surface of the first end part 16a that is confronted with the cylinder 10. The second adhesive layer 24 is provided on one surface of the second end part 16b of the swab pack 16, viz., on the surface of the second end part 16b that faces the cylinder 10 when the swab pack 16 is wound around the cylinder 10. The first and second adhesive layers 22, 24 may be a coat of adhesive materials such as epoxy resin or a commercially available double side tape.

The swab pack 16 is removably fastened to the cylinder 10 by securing the first end part 16a of swab pack 16 to the cylinder 10 with the use of the first adhesive layer 22, winding the intermediate part of the swab pack 16 around the cylinder 10 and then bonding the second end part 16b of swab pack 16 to the intermediate part with the use of the second adhesive layer 24. Once the swab pack 16 is fastened to the cylinder 10 in this fashion, it maintains the wrapped condition unless a peeling force is applied to the second end part 16b by the syringe user.

It should be appreciated that the first adhesive layer 22 is not an essential element in the modification shown in FIGS. 3 through 5 and may be omitted, if desired. In other words, the swab pack 16 may be secured to the cylinder 10 with the second adhesive layer 24 alone.

Referring to FIG. 6, there is illustrated a modified swab pack that can be employed in the disposable syringe illustrated in FIG. 3. This swab pack 16 has the same structure and shape as the one shown in FIGS. 3 through 5 except that an adhesive layer 26 is formed on the entire area of one surface of the swab pack 16. This type of swab pack 16 can be wrapped around and kept in place on the cylinder 10 with a greater bonding strength.

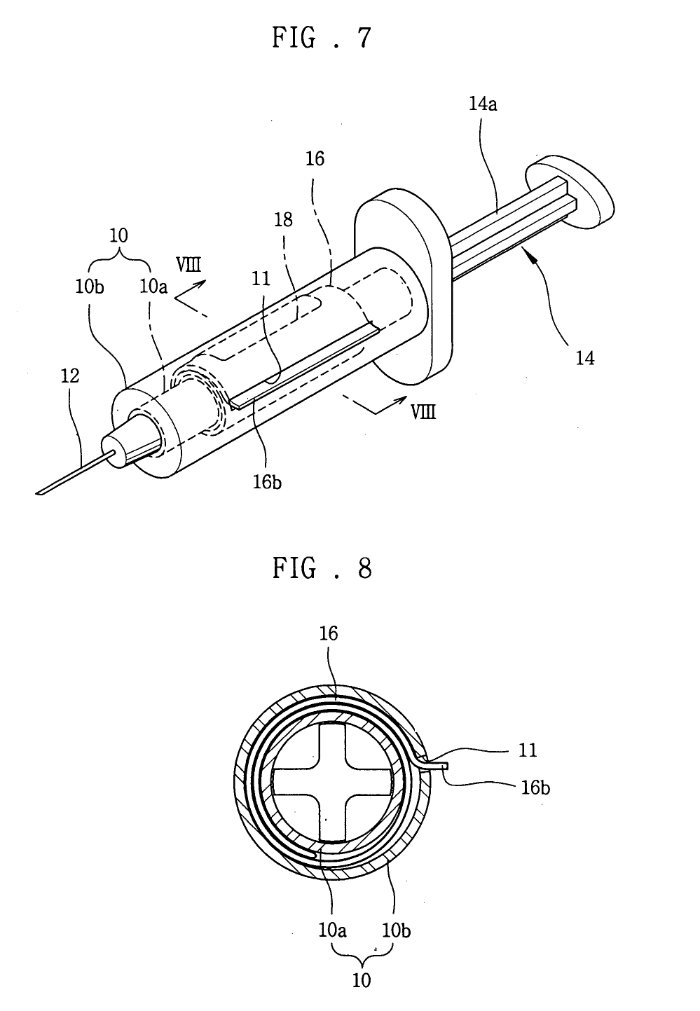

With reference to FIGS. 7 and 8, there is illustrated another modification of the disposable syringe according to the first embodiment of the present invention. Unlike in the preceding embodiment and modification, the disposable syringe is provided with a cylinder 10 that consists of an inner cylinder part 10a and an outer cylinder part 10b surrounding the inner cylinder part 10a in a spaced-apart coaxial relationship.

The outer cylinder part 10b has a slit 1 extending along an axis of the cylinder 10 in a length a little greater than the width of the swab pack 16. An annular gap is left between the inner and outer cylinder parts 10a, 10b. The annular gap serves as a fastener means for the swab pack 16 and receives the swab pack 16 with one end part thereof exposed to the outside through the slit 11 of the outer cylinder part 10b. When in use, the swab pack 16 can be taken out from the annular gap of the cylinder 10 by grasping and pulling the exposed end of the swab pack 16.

Other parts and components of the disposable syringe according the present modification are the same as those of the first embodiment shown in FIGS. 1 and 2 and therefore designated by the same reference numerals with no description offered in that regard.

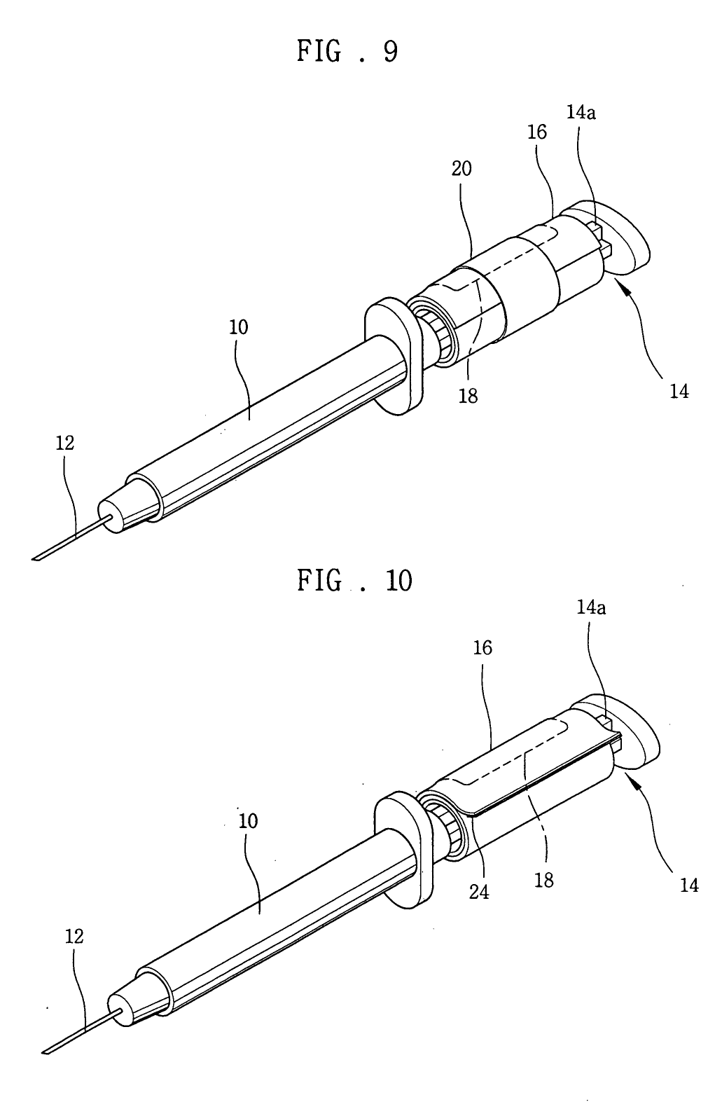

Referring to FIG. 9, there is shown a disposable syringe with a swab pack according to a second embodiment of the present invention. As with the first embodiment and its modifications set forth earlier, the disposable syringe is composed of a hollow cylinder 10 for containing liquid medication, e.g., insulin solution. A needle 12 extends forwards from the frontal end of the cylinder 10 and provides a flow path through which the liquid medication is injected into the body of a syringe user. A plunger 14 is slidably fitted to the cylinder 10 at an opposite side from the needle 12 in a liquid-tight manner. The plunger 14 has a grip 14a that can be grasped by the syringe user. The plunger 14 is movable between a retracted position in which the liquid medication is injected through the needle 12 and an extended position in which the liquid medication is drawn into the cylinder 10.

Attention should be drawn to the fact that, in the present embodiment, the grip 14a of the plunger 14 removably holds a hermetically sealed flexible swab pack 16 that contains a swab 18 dipped with antiseptic liquid such as alcohol. The swab pack 16 is made of, e.g., a synthetic resin film with air and liquid impermeability and hermetically sealed to avoid volatilization and leakage of the antiseptic liquid impregnated into the swab 18. The swab pack 16 is readily torn by the syringe user so that the swab can be taken out when needed, namely, when the body spot is to be rubbed with the swab for pre-injection and post-injection disinfection.

A fastener means is employed to keep the swab pack 16 in place on the grip 14a of the plunger 14 against inadvertent removal. A sheet sleeve 20 serves as the fastener means in the second embodiment shown in FIG. 9 and is adapted to encircle the swab pack 16 wound around the grip 14a of the plunger 14 to thereby keep the swab pack 1.6 in place. The sheet sleeve 20 is made of easy-to-tear materials, e.g., paper, such that the syringe user can tear away the sheet sleeve 20 in order to separate the swab pack 16 from the plunger 14.

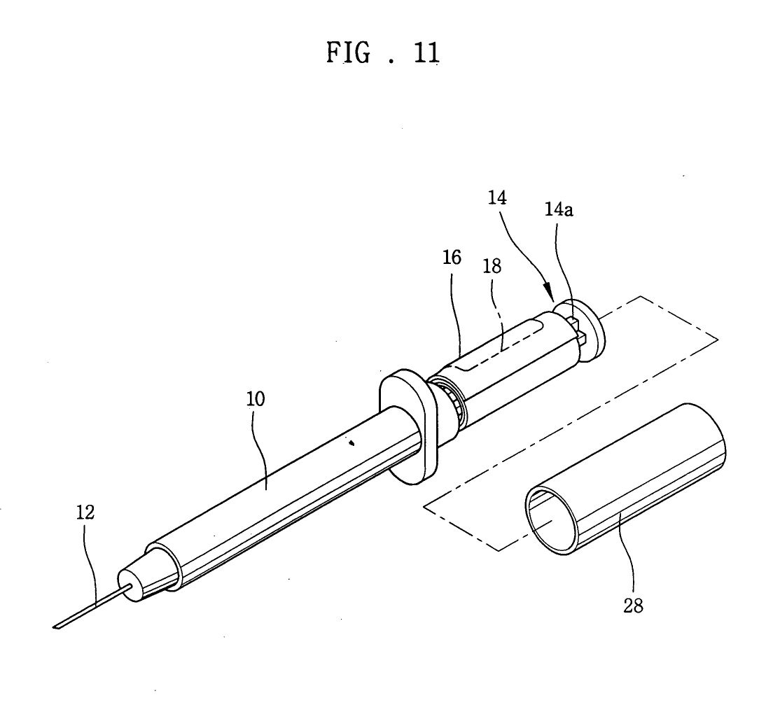

FIG. 10 shows one modification of the disposable syringe according to the second embodiment of the present invention. In this modification, the grip 14a of the plunger 14 removably holds a hermetically sealed flexible swab pack 16 that contains a swab 18 dipped with antiseptic liquid such as alcohol. Other parts and components of the disposable syringe according the present modification are the same as those of the second embodiment shown in FIG. 9 and therefore designated by the same reference numerals with no description offered in that regard.

A first adhesive layer 22 and a second adhesive layer 24 serve as the fastener means for keeping the swab pack 16 in place. The first adhesive layer 22 is formed on one surface of the first end part 16a of the swab pack 16, viz., on the surface of the first end part 16a that is confronted with the grip 14a of the plunger 14. The second adhesive layer 24 is provided on one surface of the second end part 16b of the swab pack 16, viz., on the surface of the second end part 16b that faces the plunger 14 when the swab pack 16 is wound around the plunger 14. The first and second adhesive layers 22, 24 may be a coat of adhesive materials such as epoxy resin or a commercially available double side tape.

The swab pack 16 is removably fastened to the grip 14a of the plunger 14 by securing the first end part 16a of swab pack 16 to the grip 14a with the use of the first adhesive layer 22, winding the intermediate part of the swab pack 16 around the grip 14a and then bonding the second end part 16b of swab pack 16 to the intermediate part with the use of the second adhesive layer 24. Once the swab pack 16 is fastened to the plunger 14 in this fashion, it maintains the wrapped condition unless a peeling force is applied to the second end part 16b by the syringe user.

FIG. 11 shows another modification of the disposable syringe according to the second embodiment of the present invention, wherein an end cap is used as a fastener means for a swab pack. Other parts and components of the disposable syringe according the present modification are the same as those of the second embodiment shown in FIG. 9 and therefore designated by the same reference numerals with no description offered in that regard.

In this modification, the swab pack 16 is simply wrapped around the grip 14a of the plunger 14 and an elongated end cap 28 is fitted to the grip 14a from the terminal end thereof in such a manner that the end cap 28 can enclose the swab pack 16 substantially for the most part. This means that the end cap 28 functions as a fastener means for the swab pack 16. Removing the end cap 28 exposes the swab pack 16 to the outside and places it in condition for use.

Although certain preferred embodiments of the present invention have been described in detail, it will be apparent to those skilled in the art that various changes or modifications may be made thereto within the scope of the invention defined by the appended claims.

What is claimed is:

1. A disposable syringe, comprising:
a hollow cylinder for containing liquid medication;
a needle extending forwards from a frontal end of the cylinder;
a plunger slidably fitted to the cylinder at an opposite side from the needle in a liquid-tight manner;
a hermetically sealed flexible swab pack containing antiseptic liquid-dipped swab, the swab pack removably held by the cylinder; and
a fastener means for keeping the swab pack in place on the cylinder against inadvertent removal.

2. The disposable syringe as recited in claim 1, wherein the swab pack is wound around the cylinder in a spiral shape and having a first end part in contact with the cylinder and a second end part lying over an intermediate extension of the swab pack.

3. The disposable syringe as recited in claim 2, wherein the fastener means comprises a sheet sleeve encircling the swab pack to prevent the swab pack from unwinding.

4. The disposable syringe as recited in claim 1, wherein the fastener means comprises a sheet sleeve encircling the cylinder and the swab pack.

5. The disposable syringe as recited in claim 2, wherein the fastener means comprises an adhesive layer provided on one surface of the second end part of the swab pack.

6. The disposable syringe as recited in claim 5, wherein the fastener means further comprises an adhesive layer provided on one surface of the first end part of the swab pack.

7. The disposable syringe as recited in claim 1, wherein the fastener means comprises an adhesive layer provided substantially on the entire area of one surface of the swab pack.

8. The disposable syringe as recited in claim 1, wherein the cylinder is provided with an inner cylinder part and an outer cylinder part surrounding the inner cylinder part in a spaced-apart coaxial relationship, the outer cylinder part having a slit extending along an axis of the cylinder.

9. The disposable syringe as recited in claim 8, wherein the fastener means comprises an annular gap left between the inner and outer cylinder parts, the swab pack received in the annular gap with one end part thereof exposed to the outside through the slit of the outer cylinder part.

10. A disposable syringe, comprising:
a hollow cylinder for containing liquid medication;
a needle extending forwards from a frontal end of the cylinder;
a plunger slidably fitted to the cylinder at an opposite side from the needle in a liquid-tight manner and having a grip exposed to the outside from the cylinder;
a hermetically sealed flexible swab pack containing antiseptic liquid-dipped swab, the swab pack removably held by the grip of the plunger; and
a fastener means for keeping the swab pack in place on the grip of the plunger against inadvertent removal.

11. The disposable syringe as recited in claim 10, wherein the swab pack is wound around the grip of the plunger in a spiral shape and having a first end part in contact with the grip and a second end part lying over an intermediate extension of the swab pack.

12. The disposable syringe as recited in claim 11, wherein the fastener means comprises a sheet sleeve encircling the swab pack to prevent the swab pack from unwinding.

13. The disposable syringe as recited in claim 11, wherein the fastener means comprises an adhesive layer provided on one surface of the second end part of the swab pack.

14. The disposable syringe as recited in claim 13, wherein the fastener means further comprises an adhesive layer provided on one surface of the first end part of the swab pack.

15. The disposable syringe as recited in claim 10, wherein the fastener means comprises an adhesive layer provided substantially on the entire area of one surface of the swab pack.

16. The disposable syringe as recited in claim 11, wherein the fastener means comprises an end cap removably fitted to the grip of the plunger for enclosing the swab pack wound around the grip.
